(19) United States
(12) Patent Application Publication
(10) Pub. No.: US 2005/0050768 A1
(43) Pub. Date: Mar. 10, 2005

(54) Orthotic sandal

(76) Inventors: Johan Louis Fehrensen, La Jolla, CA (US); Stephanie Hoffman, La Jolla, CA (US)

Correspondence Address: LOUIS FEHRENSEN, 2510 TORREY PINES RD. #215, LA JOLLA, CA, 92037, US

(21) Appl. No.: 10/659,209

(22) Filed: Sep. 9, 2003

(51) Int. Cl. A43B 3/12; A61F 5/14

(52) U.S. Cl. 36/11.5

(57) ABSTRACT

A shoe for accommodating the normal mechanics of the foot and establishing a predetermined shape and degree of inclination for the rear foot bed includes a sandal incorporating layers of different materials in a predetermined order. One of the layers is a wedge-shaped layer that is shaped to provide an incline of between 2 and 6 degrees relative to a horizontal ground plane. The other layers of the sandal are stacked with the wedge-shaped layer to create an inclined rear foot bed. In operation, the user predetermines the angle of tilt between 2 and 6 degrees which will anatomically accommodate and correct his foot ailment and, accordingly, chooses a sandal having the preselected incline.

### FIELD OF THE INVENTION

The present invention pertains generally to devices and methods which are useful for reducing foot, ankle, knee, hip and lower back strain. More particularly, the present invention pertains to devices and methods of using same which employ techniques for encouraging the normal mechanics of the foot. The present invention is particularly, but not exclusively, useful as a biomechanical orthotic sandal where the foot is aligned in a substantially neutral position and where excessive foot motion is controlled during exercise or other normal activity.

### BACKGROUND OF THE INVENTION

The general principles of biomechanical treatment of the foot for the challenging problem of excessive pronation are well known and well understood. In short, a biomechanical orthotic device used as a method of foot treatment aligns the foot in a substantially neutral position which will cause the normal mechanics of supination and pronation. Typically in the prior art, orthotic devices that are placed inside a shoe have been implemented to create an environment which controls excessive motion. Such conditions not only allow the body to operate at a greater efficiency, but repetitive foot strain is also significantly reduced.

In the field of biomechanical orthotics, the object is to encourage normal mechanics of the foot. Specifically, the object is to protect the foot, ankle, knee, hip and the low back region from undue strain and tension generated by the ground reaction forces being transferred in an upwards direction from the feet to the knee, hip and back region during motion. The amount of generated force and strain varies with several factors. These factors may include: the ground surface material, a person's age, a person's current state of health, the structural integrity of the shoe, type of activity, and quantity of training.

Various devices and techniques have developed over recent years to correct individual foot problems. Of particular interest here are the so-called orthotic inserts that can biomechanically correct a specific diagnosed problem. An orthotic insert is an inclined ridge that provides support to the affected portions of the foot when it is placed inside the shoe. Foot correction is achieved in main part by this added support. Prior art, however, does not offer a sandal which is specifically designed to include an orthotic insert with standardized degrees of correction.

Orthotic insert correction, though very effective, is limited by the physical characteristics of the orthotic insert. Typically, since an orthotic insert does not tightly fasten to the shoe, slippage occurs. With slippage comes the valid concern that the insert may not be functioning properly and the insert is not accomplishing its purpose.

The present invention recognizes the concerns mentioned above. It also recognizes that sandals are a form of shoe that are desired by people and highly advantageous to them for other reasons. Specifically, sandals are desirous to people in warm climates since a sandal is designed to lend adequate air circulation. Through this added air circulation, a sandal provides comfort, which in turn improves the quality of a person's life.

In addition, it is important to people involved in sports to carefully select a shoe which will help them achieve optimum performance in a given sport. In regard to this consideration, the sandal is the ideal shoe for certain water sport activities. A shoe such as one envisioned for the present invention, that both accommodates certain water sports activities and enables biomechanical correction, is preferred by users that play such sports during their quest to maintain their active lifestyle while correcting their foot problems.

Many people are troubled by foot problems because they regard them as intrusive upon other aspects of their life such as personal style. The prior art solutions are not aesthetically pleasing to everyone as they are visible and bulky. Furthermore, some people feel their shoe wardrobe is limited due to a lack of variety of corrective shoes offered in the market today. By offering a corrective sandal type of shoe, people will have a broader selection of types of shoes to choose from and people will not feel that their foot problems are intruding upon aspects of their life, even those unrelated to health-be that as it may, such as personal style.

Accordingly. and in light of the above, it is an object of the present invention to provide a sandal which will dissipate ground forces generated in the foot region. It is another object of the present invention to provide a sandal which eliminates slippage of an orthotic insert. It is yet another objective to provide a sandal that corrects biomechanical abnormalities. Still another objective of the present invention is to provide a sandal, and a method of manufacturing same which is easy to implement and comparatively cost effective.

### SUMMARY OF THE INVENTION

A method and device in accordance with the present invention, includes a shoe in the form of a sandal to treat excessive pronation of the foot wherein the user predetermines the heel tilt angle in the range of 2 and 6 degrees that is suitable for correcting his specific ailment. In overview, the methods and products that are disclosed for the present invention involve the combination of various layers of materials in the creation of a sandal with an inclined heel foot bed area.

An orthotic sandal comprises an assembly consisting of a series of layers and a wedge-shaped structure that is oriented at an angle α in the range of 2-6 degrees from the latitudinal x-axis of the ground plane. The first layer, the top sole layer, has a substantially flat surface and may be made of foam rubber or it may be made of a moldable material such as a high density rubber. Further, the top sole layer may be coated, or uncoated, or composed of a different lightweight material. The top sole layer is placed directly above the top surface of the wedge-shaped structure.

A second layer, referred to as the bottom sole layer, is also formed into the sandal. Like the top sole layer, it has a substantially flat surface that may be made of foam rubber or it may be made of a moldable material such as a high density rubber. Where this layer is concerned, a coating or covering may also be applicable. The lower bottom sole layer can also be uncoated. Preferably, this layer is attached or pressed against the wedge-shaped structure bottom surface.

Alternatively, a plurality of other layers can be bonded to the above-stated two layers and wedge layer. For example, an upper sole covering can be bonded to the top surface of the upper sole layer. Likewise, a bottom covering can be bonded to the bottom surface of the lower bottom sole layer. Shock absorbency is increased by increasing the number of layers. The consequence here is that although shock absorbency is increased, the weight of the shoe may also increase and there may be a decrease in aesthetic value. In all cases, the trade-off between the quality of foot treatment and the increase in shoe weight and a risk in the decrease in shoe aesthetic value are important considerations when establishing the number and types of layers of an orthotic sandal.

As envisioned for the present invention, the orthotic sandals efficaciously resist rotational movement and vertical displacement of the corrective orthotic wedge-shaped structures following the insertion of the user's feet.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of the sandal for a right foot in accordance with the present invention with portions broken away for clarity;

FIG. 2 is a perspective view of an alternative embodiment of the sandal for a right foot with portions broken away for clarity;

FIG. 3 is a rear face view of the sandal for a right foot in accordance with the present invention;

FIG. 4 is a Cartesian coordinate system describing the angle β as used for the present invention; and

FIG. 5 is a perspective schematic view showing the relationship, as angle β, between a ground surface and the modular wedge as used for the present invention.

### DETAILED DESCRIPTION OF THE PREFERRED EMBODIMENTS

In recognition of the above-described difficulties with prior art foot treatments and orthotic inserts, the present disclosure describes a device and method for treating and correcting excessive foot pronation. The device described below includes a shoe appropriately configured as a sandal that substantially eliminates excessive pronation of the foot. Consequently for purposes of illustration, the exemplary embodiments of the invention are described in a manner consistent with such configuration, though clearly the invention is not so limited.

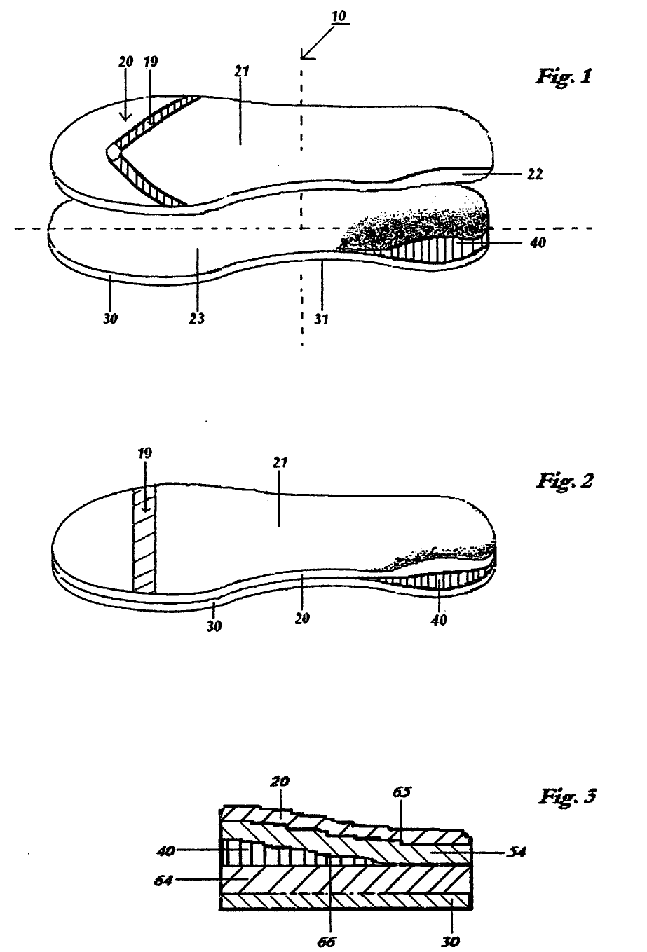

Referring initially to FIG. 1 and FIG. 2, an orthotic sandal 10 is shown in accordance with the current invention. The advantages of the present device and method include the increase in efficiency of the body, specifically the foot, ankle, knee, hip and lower back region and a reduction of repetitive strain during motion by bringing the foot into a biomechanically optimal position. This may provide a significant advantage in applications such as water sports and everyday walking for people who have active lifestyles, who seek comfortable shoes and need to correct a foot ailment.

The orthotic sandal 10 essentially includes a top sole layer 20 with a top surface 21 that has a relatively flat surface which extends across the length of the sole of a foot. The top sole layer 20 includes a strap 19 that may have different variations and embodiments. The strap 19 is used to secure the foot to the sandal. The bottom surface 22 of the top sole layer 20 is positioned against the top surface 23 of a bottom sole layer 30. The bottom sole layer 30 has a surface 31 opposing the top surface 23 which the user places against a ground surface or within a fluid in motion. Additionally, there is a wedge-shaped structure 40 which is included between the top sole layer 20 and bottom sole layer 30. The wedge-shaped structure 40 is placed near the heel area 42 of the sandal 10 in order to correct a foot problem of excessive pronation.

For purposes of this invention, the wedge-shaped structure 40 may be made of different types of suitable materials known in the pertinent art which are lightweight, moldable and of a high density.

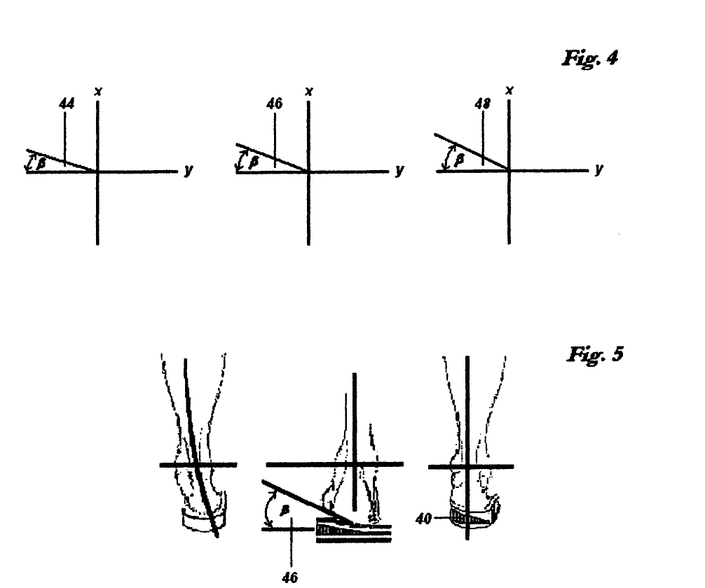

Regardless of the material used for wedge-shaped structure 40, the wedge-shaped structure 40 will preferably be shaped as a rectangular or square wedge having preferably one slope which inclines the sole of the foot. Alternatively, the wedge-shaped structure 40 can be made of a different shape such as a circle. In any case, the wedge-shaped structure 40 will include a slope. With reference to FIG. 4, this invention envisions angle of the slope, angle β41 from a ground plane surface 42, of the wedge-shaped structure to preferably be between 2 and 6 degrees. For example, as shown in FIG. 4 in an exaggerated view, a sandal can have a slope of two degrees 44, four degrees 46 or six degrees 48. As best seen in FIG. 5, the wedge-shaped structure 40 causes the foot to be in an inclined position from a ground plane when a foot is inserted in a sandal. Importantly, the wedge-shaped structure 40 must be capable of being varied in orientation both within the layers and about the foot bed area. However, the wedge-shaped structure 40 must not shift after it is placed within the sandal construction. Preferably, the wedge-shaped structure 40 is kept in place either by force from the other layers or a bonding method.

FIG. 3 shows that the sandal 10 can include additional layers such as an upper middle layer 54 and a lower middle layer 64. The upper middle layer 54 has a top surface 65 and a bottom surface 66.

The top surface 65 is placed against the bottom 22 surface of the top sole layer 20. The bottom surface 66 is placed against the wedge-shaped structure 40. Similarly, the lower middle layer 64 is placed between the wedge-shaped structure 40 and the bottom sole layer 30.

In operation, the user first determines what degree of incline is necessary for correcting his foot ailment. The user then selects a sandal having a wedge-shaped structure with a tilt angle β of between 2 and 6 degrees.

While specific embodiments of the invention have been illustrated and described, other embodiments and variations are possible. For example, the sandal strap as illustrated in FIG. 1 can be substituted with a front, back, left side and right side panel that provides more coverage than a single or double strap.

All these are intended to be encompassed by the following claims.

1. A sandal for creating an inclination from the inner portion of the heel to the outer heel portion to adjust the axial canting of the bottom surface of the foot relative to the horizontal walking surface and to bring the foot into a biomechanically optimal position, said sandal comprising: a bottom sole layer having a top surface, a bottom surface, and a heel area; an upper middle sole layer having a top surface, a bottom surface, and a heel area; a lower middle sole layer having a top surface, a bottom surface, and a heel area; a top sole layer having a top surface and a bottom surface and a heel area; and a wedge-shaped structure, wherein said wedge-shaped structure is located between said upper middle sole layer and said lower middle sole layer near the heel area of said upper middle sole layer and said lower middle sole layer to inhibit movement of said wedge-shaped structure in said sandal, said structure defining a tilt angle α between 2 degrees and 6 degrees relative to a substantially flat horizontal walking plane.

2. The sandal of claim 1, wherein said top sole includes a strap for securing said sandal to a foot.

3. The sandal of claim 1, wherein the top sole includes a Velcro strap for securing said sandal to a foot.

4. The sandal of claim 1, wherein the top sole includes a strap having a buckle clasp for securing said sandal to a foot.

5. The sandal of claim 1, wherein the top sole includes a right elongated loop strap and a left elongated loop strap for a thong style shoe.

6. The sandal of claim 1, wherein said bottom sole is made of a moldable material.

7. The sandal of claim 6, wherein said moldable material is made from rubber, said rubber having a high density.

8. The sandal of claim 1, wherein said wedge-shaped structure is fixedly attached to said top sole and said bottom sole.

9. The sandal of claim 1, wherein said wedge-shaped structure is removably wedged between said top sole and said bottom sole.

10. The sandal of claim 1, wherein the material of said bottom surface of said bottom sole is a wear resistant material.

11. The sandal of claim 1, wherein said sandal further comprises a plurality of sublayers positioned against said top sole layer.

12. The sandal of claim 1, wherein said sandal further comprises a plurality of sublayers positioned against said bottom sole layer.

13. The sandal of claim 1, wherein said sandal further comprises a plurality of sublayers positioned against said upper middle sole layer.

14. The sandal of claim 1, wherein said sandal further comprises a plurality of sublayers positioned against said lower middle sole layer.

15. The sandal of claim 1, wherein said sandal further comprises a hollow shoe covering located on top of said top sandal layer, said shoe covering having an opening wherein a foot can be inserted and a top side panel, left side panel, right side panel, and back side panel.

16. A method for bringing the foot into a biomechanically optimal position, the method comprising: providing a sandal having a bottom sole layer having a top surface, a bottom surface, and a heel area, an upper middle sole layer having a top surface, a bottom surface, and a heel area, a lower middle sole layer, a top sole layer having a top surface and a bottom surface and a heel area, and a wedge-shaped structure, said wedge-shaped structure aligned between said upper middle sole layer and said lower middle sole layer near heel area of said upper middle sole layer and said bottom middle sole layer to inhibit movement of said wedge-shaped structure in said sandal, said structure defining a tilt angle β between 2 degrees and 6 degrees relative to a substantially flat horizontal walking plane; and placing a foot in contact with said top surface of said top sole layer.

17. A method as recited in claim 16, wherein said top sole layer includes a strap for securing said sandal to a foot.

18. A method as recited in claim 16, wherein the top sole layer includes a Velcro strap for securing said sandal to a foot.

19. A method as recited in claim 16, wherein the top sole layer includes a strap having a buckle clasp for securing said sandal to a foot.

20. A method as recited in claim 16, wherein the top sole layer includes a right elongated loop strap and a left elongated loop strap for a thong style shoe.

21. A method as recited in claim 16, wherein the bottom sole layer is made of a moldable material.

22. A method as recited in claim 16, wherein the moldable material is made from rubber, said rubber having a high density.

23. A method as recited in claim 16, wherein said wedge-shaped structure is fixedly attached to said top sole layer and said bottom sole layer.

24. A method as recited in claim 16, wherein the material of said bottom surface of said bottom sole is a wear resistant material.

25. The method as recited in claim 16, wherein said sandal further comprises a hollow shoe covering located on top of said top sandal layer, said shoe covering having an opening wherein a foot can be inserted and a top side panel, left side panel, right side panel, and back side panel.

26. The method as recited in claim 16, wherein said sandal further comprises a plurality of sublayers positioned against said bottom sole layer.

27. The method as recited in claim 16, wherein said sandal further comprises a plurality of sublayers positioned against said top sole layer.

28. The method as recited in claim 16, wherein said sandal further comprises a plurality of sublayers positioned against said upper middle layer.

29. The method as recited in claim 16, wherein said sandal further comprises a plurality of sublayers positioned against said lower middle layer.

30. A sandal for creating an inclination from the inner portion of the heel to the outer heel portion to adjust the axial canting of the bottom surface of the foot relative to the horizontal walking surface and to bring the foot into a biomechanically optimal position, said sandal comprising: a bottom sole layer having a top surface, a bottom surface, and a heel area; a top sole layer having a top surface and a bottom surface and a heel area; and a wedge-shaped structure, wherein said wedge-shaped structure is located between said bottom sole and said top sole near the heel area of said bottom sole and said top sole to inhibit movement of said wedge-shaped structure in said sandal, said structure defining a tilt angle β between 2 degrees and 6 degrees relative to a substantially flat horizontal walking plane.

31. The sandal of claim 30, wherein said top sole includes a strap for securing said sandal to a foot.

32. The sandal of claim 30, wherein the top sole includes a velcro strap for securing said sandal to a foot.

33. The sandal of claim 30, wherein the top sole includes a strap having a buckle clasp for securing said sandal to a foot.

34. The sandal of claim 30, wherein the top sole includes a right elongated loop strap and a left elongated loop strap for a thong style shoe.

35. The sandal of claim 30, wherein said bottom sole is made of a moldable material.

36. The sandal of claim 30, wherein said moldable material is made from rubber, said rubber having a high density.

37. The sandal of claim 30, wherein said wedge-shaped structure is fixedly attached to said top sole and said bottom sole.

38. The sandal of claim 30, wherein said wedge-shaped structure is removably wedged between said top sole and said bottom sole.

39. The sandal of claim 30, wherein the material of said bottom surface of said bottom sole is a wear resistant material.

40. The sandal of claim 30, wherein said sandal further comprises a plurality of sublayers positioned against said top sole layer.

41. The sandal of claim 30, wherein said sandal further comprises a plurality of sublayers positioned against said bottom sole layer.

42. The sandal of claim 30, wherein said sandal further comprises a hollow shoe covering located on top of said top sandal layer, said shoe covering having an opening wherein a foot can be inserted and a top side panel, left side panel, right side panel, and back side panel.

43. A method for bringing the foot into a biomechanically optimal position, the method comprising: providing a sandal having a bottom sole layer having a top surface, a bottom surface, and a heel area, a top sole layer having a top surface and a bottom surface and a heel area, a wedge-shaped structure, said wedge-shaped structure aligned between said bottom sole layer and said top sole layer near heel area of said bottom sole layer and said top sole layer to inhibit movement of said wedge-shaped structure in said sandal, said structure defining a tilt angle β between 2 degrees and 6 degrees relative to a substantially flat horizontal walking plane; and placing a foot in contact with said top surface of said top sole layer.

44. A method as recited in claim 43, wherein said top sole layer includes a strap for securing said sandal to a foot.

45. A method as recited in claim 43, wherein the top sole layer includes a Velcro strap for securing said sandal to a foot.

46. A method as recited in claim 43, wherein the top sole layer includes a strap having a buckle clasp for securing said sandal to a foot.

47. A method as recited in claim 43, wherein the top sole layer includes a right elongated loop strap and a left elongated loop strap for a thong style shoe.

48. A method as recited in claim 43, wherein the bottom sole layer is made of a moldable material.

49. A method as recited in claim 43, wherein the moldable material is made from rubber, said rubber having a high density.

50. A method as recited in claim 43, wherein said wedge-shaped structure is fixedly attached to said top sole layer and said bottom sole layer.

51. A method as recited in claim 43, wherein the material of said bottom surface of said bottom sole is a wear resistant material.

52. The method as recited in claim 43, wherein said sandal further comprises a hollow shoe covering located on top of said top sandal layer, said shoe covering having an opening wherein a foot can be inserted and a top side panel, left side panel, right side panel, and back side panel.

53. The method as recited in claim 43, wherein said sandal further comprises a plurality of sublayers positioned against said bottom sole layer.

54. The method as recited in claim 43, wherein said sandal further comprises a plurality of sublayers positioned against said top sole layer.
